(19) United States
(12) Patent Application Publication
(10) Pub. No.: US 2003/0176798 A1
(43) Pub. Date: Sep. 18, 2003

(54) Method and device for detecting cardiac arrest and automatically alerting emergency personnel of wearer's location

(76) Inventor: Arnold Baruch Simon, Stuart, FL (US)

Correspondence Address: FLEIT KAIN GIBBONS GUTMAN & BONGINI, COURVOISIER CENTRE II, SUITE 404, 601 BRICKELL KEY DRIVE, MIAMI, FL, 33131, US

(21) Appl. No.: 10/364,916

(22) Filed: Feb. 11, 2003

Related U.S. Application Data
(60) Provisional application No. 60/356,016, filed on Feb. 12, 2002.

(51) Int. Cl. A61B 5/0402

(52) U.S. Cl. 600/509

(57) ABSTRACT

A method, a system and a computer readable medium for detecting cardiac arrest and automatically alerting emergency personnel of a wearer's location, include detecting at least one dangerous heart abnormality based on measuring a user's current pulse by a first module, and generating an emergency signal to trigger a second wireless module. The wireless module emits an audible alarm for a given time, and if the alarm is not disabled within the given time, the wireless module initiates an emergency call for determining the geographic coordinates of the wireless module. The method further includes transmitting a predefined message after initiating the emergency call.

### CROSS REFERENCE TO RELATED APPLICATION

The benefit of Provisional U.S. Patent Application No. 60/356,016, filed Feb. 12, 2002, is claimed under 35 USC §119(e). The contents of this application are hereby incorporated by reference.

### FIELD OF THE INVENTION

The present invention generally relates to the field of remote monitoring of vital signs, and more particularly relates to a method and a wireless heart beat monitor for automatically calling 911 during ventricular fibrillation or heart stoppage to alert emergency personnel of the wearer's location.

### BACKGROUND OF THE INVENTION

Phase H of the Federal Communications Committee's Enhanced 911 (E911) mandate requires wireless carriers, including cellular licensees, broadband Personal Communications Service (PCS) licensees, and certain Specialized Mobile Radio (SMR) licensees, to provide Automatic Location Identification (ALI) as part of Phase II E911 implementation beginning Oct. 1, 2001. These carriers must modify their handsets and/or networks to meet the following location accuracy:

For handset-based solutions: 50 meters for 67 percent of calls, 150 meters for 95 percent of calls; and

For network-based solutions: 100 meters for 67 percent of calls, 300 meters for 95 percent of calls.

Since 1996, 911 calls can be placed on a wireless device without charge and or requirement of a service plan from any provider. Wireless 911 calls can be made on analog cellular phone or dual or tri-mode phones anywhere there is an analog network. However, before Phase II of E911, callers must provide the emergency operator their location and situation. At least two manufacturers, Motorola and Magnavox, produce 911-only phones that do not require subscription to a carrier.

The Global Positioning System (GPS) was setup by the US defense department and was made available for civilian use in 1990. Through the use of signals of three satellites of a 21-satellite constellation, a GPS receiver can triangulate its position with 15 meters root mean square accuracy.

However, a problem with E911 is that it provides only 50-300 meters location accuracy. Thus, E911 is unsuitable for use in a multi-person building. In other words, a person's location (in a multi-person building) with an emergency cannot be accurately detected using E911. Accordingly, a need exists to provide a way to accurately detect a person's location in an emergency situation.

Another shortcoming with the related art is that, where E911 is unavailable, the location of the person with an emergency is not identifiable. Thus, a cell phone cannot be used to detect the location in that context. Therefore, in an emergency situation, a need exists to provide a way to identify a person's location notwithstanding the fact that E911 is unavailable.

Still another shortcoming with the current devices is that they do not provide a localized alarm. For instance, a person having a cardiac arrest and just wearing a heart rate monitor does not automatically notify (of the cardiac arrest) others in the vicinity. Accordingly, in an emergency situation, a need exists to provide a way to notify others in the vicinity (about the emergency).

Yet another problem with the prior art is the necessity of purchasing specialized hardware/software. However, many a people already have cell phones and prefer to use the existing cell phones. Accordingly, a need exists to dispense with the necessity of purchasing additional specialized hardware/software and to make use of the existing cell phones.

Further, there is no existing device that has a first module that generates an audible alarm, which triggers another wireless module to transmit a message if the audible alarm is not disabled within a given time. In addition, in areas where no emergency services are available, there is no conventional system which is customized for generating only the loud signal. Accordingly, a need exists to provide a method, system and a computer readable medium for overcoming the above-problems.

### SUMMARY OF THE INVENTION

Accordingly, the present invention includes a method (on a user unit), system and a computer readable medium for detecting cardiac arrest and automatically alerting emergency personnel of a wearer's location. The method includes detecting at least one dangerous heart abnormality based on measuring a user's current pulse by a first module, and generating an emergency signal to trigger a second wireless module. The wireless module emits an audible alarm for a given time, and if the alarm is not disabled within the given time, the wireless module initiates an emergency call for determining the geographic coordinates of the wireless module. The method further includes transmitting a predefined message after initiating the emergency call.

The system includes a first module for detecting at least one dangerous heart abnormality and generating an emergency signal; and a second wireless module in communication with the first module for receiving the emergency signal therefrom.

### BRIEF DESCRIPTION OF THE DRAWINGS

FIG. 1 is a diagram illustrating a person wearing a wristwatch module and a wireless phone module (worn on the belt), according to the present invention.

FIGS. 2a-2b are diagrams illustrating the wristwatch module for tracking heart rate and for instructing the wireless phone module to dial 911, with FIG. 2a showing the front view and FIG. 2b showing the rear view, according to the present invention.

FIGS. 3a-3b are diagrams illustrating the wireless phone module, with FIG. 3a showing a stand-alone unit and FIG. 3b showing the wireless phone module with an attachment for a receiver, according to the present invention.

FIG. 4 is a block diagram of the wristwatch module of FIG. 2, according to the present invention.

FIG. 5 is a flow diagram illustrating the method operating on FIG. 4, according to the present invention.

FIGS. 6a-6b are block diagrams of the wireless phone module of FIG. 3, according to the present invention.

FIG. 7 is a flow diagram illustrating the method operating on FIG. 6, according to the present invention.

### DETAILED DESCRIPTION

Using one or more of the above-standards (with existing telephony systems) allows a heart rate monitor and a wireless phone to be combined to alert emergency personnel of the location of a person whose heart has stopped or is having ventricular fibrillation: two potentially fatal situations if not treated immediately (i.e., within four minutes).

The present invention is directed to monitor people who are at risk for cardiac arrest, heart stoppage, or ventricular fibrillation and who spend any amount of time alone.

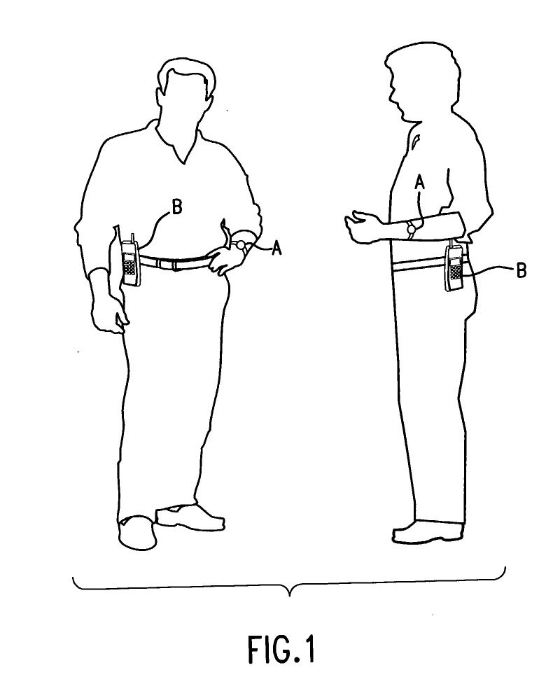

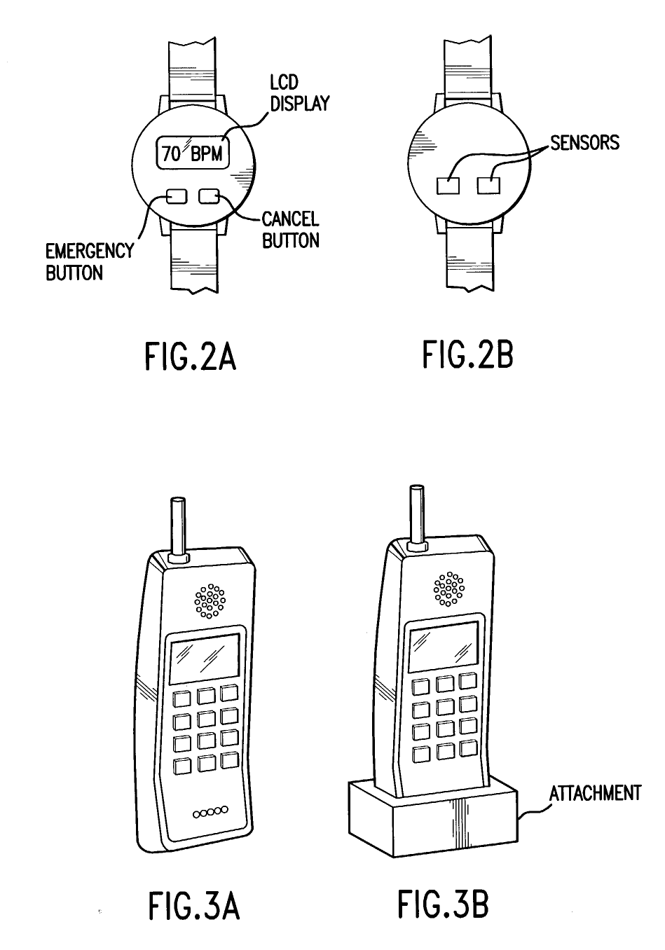

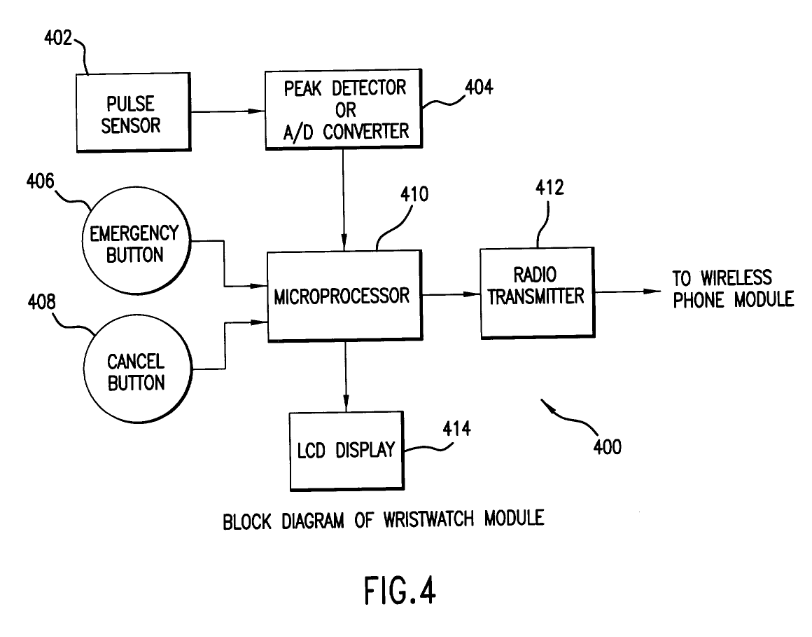

Referring now to the drawings, FIG. 1 shows the apparatus of the present invention (in one embodiment) which includes a wrist-worn device A and a wireless phone module B worn on the belt. FIG. 2 shows the wristwatch module which tracks heart rate and instructs the wireless phone module (as shown in FIG. 3) to dial 911.

Exemplary Emergency Situations

In one embodiment, the user A takes a walk by himself in the park wearing the apparatus of the present invention. In the middle of his walk, the user A suffers a heart attack and collapses unconscious. A heart rate monitor detects ventricular fibrillation or heart stoppage and generates an audible alarm to alert anyone nearby. After a predetermined time (e.g., 10 seconds), the heart monitor initiates a 911 call and transmits a prerecorded message stating an emergency situation. The 911 operator receives the phone call, and with the location information provided by E911 directs an emergency vehicle (such as an ambulance) to the general vicinity of the user A. The ambulance crew upon arriving to the scene locates the user A by the audible alarm or the crowd gathered around the user A and is able to administer emergency care (such as CPR) and take him to the hospital.

In another embodiment, a user B is a traveling businessman and stays in an out-of-town hotel room alone for the night. In this case, the minimum standard of 100 meters provided by E911 may not be enough to narrow down the room in which the businessman is staying and cost emergency personnel precious time in locating and treating him. However, a battery charger of the wireless phone is equipped with a satellite GPS receiver which is enabled when the phone is plugged into it. The phone and charger are placed near a window for better signal reception. During a detected emergency, the wireless phone retrieves the location coordinates from the GPS receiver and transmits them in the 911 voice message.

In either situation, the present invention does not require any secondary service provider to operate, since “911” is directly dialed by the device. During non-emergency situations, the wristwatch can display current heart rate information as well as basic watch features (such as time and date). The wireless phone module can be used as a traditional cellular or PCS phone provided that it is compatible with a subscribed service network.

Hardware/Software Embodiments

According to one embodiment, the present includes two units: a wristwatch (first) module 100 and a wireless phone (second) module 600. The primary functions of the wristwatch module include the detection of the heartbeats, the computation, display and monitoring of heart rate, and generation of an emergency signal to the wireless phone module.

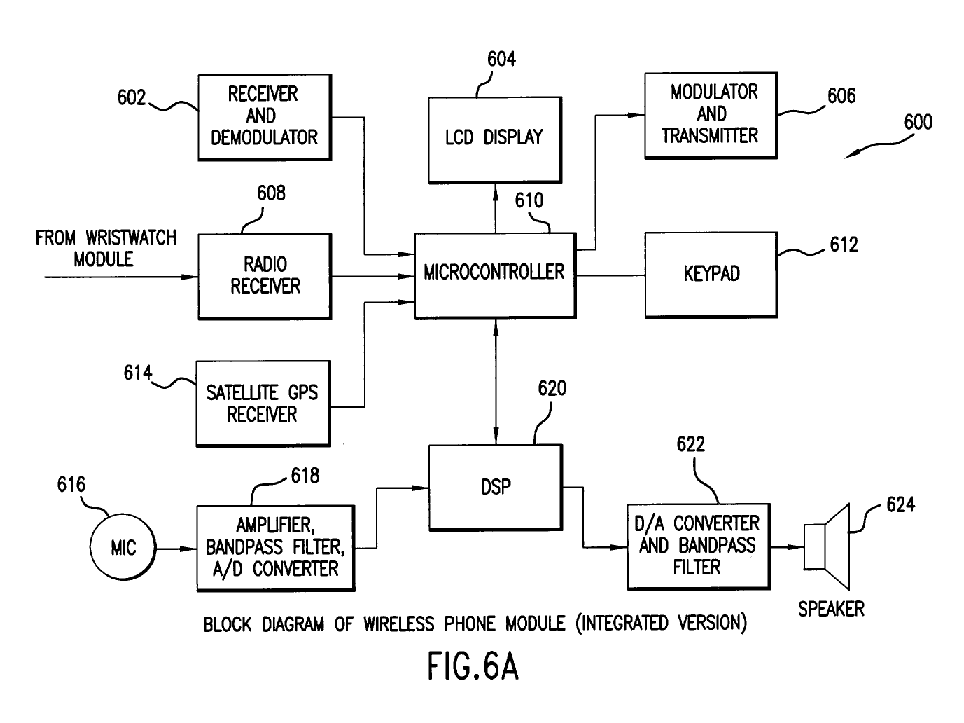

The wristwatch module 400 includes the following components: pulse sensing circuit, microprocessor, LCD display, wireless transmitter, battery, buttons, and wrist strap. The interoperability of the components is shown in FIG. 5. In addition to heart monitoring features detailed below, the wristwatch has basic time and date keeping functions.

The wireless phone module 600 combines the features of a standard cellular or PCS phone with a radio receiver 608 that receives the emergency signal from the wristwatch module 400. In one embodiment, the interfaces available in existing models of wireless phones could be connected to another unit to add the additional necessary functions. The interfaces include Bluetooth wireless interfaces, serial infrared communications interface (“SIR”), Magic Beam and other low power small distance solutions.

For instance, Bluetooth is a computing and telecommunications industry specification that describes how mobile phones, computers, and personal digital assistants (PDAs) easily interconnect with each other and with home and business phones and computers using a short-range wireless connection. Using this technology, users of cellular phones, pagers, and personal digital assistants such as the PalmPilot are able to buy a three-in-one phone that doubles as a portable phone at home or in the office, gets quickly synchronized with information in a desktop or notebook computer, initiates the sending or receiving of a fax, initiates a print-out. In general, Bluetooth coordinates the mobile and fixed computer devices.

Referring still to Bluetooth, the tranceiver transmits and receives in a previously unused frequency band of 2.45 GHz that is available globally (with some variation of bandwidth in different countries). In addition to data, up to three voice channels are available. Each device has a unique 48-bit address from the IEEE 802 standard. Connections are point-to-point and/or multipoint. The maximum range is 10 meters. Data are exchanged at up to a rate of 1 megabit per second (up to 2 Mbps in the second generation of the technology). A frequency hop scheme allows devices to communicate even in areas with a great deal of electromagnetic interference. Built-in encryption and verification is provided.

The serial infrared communications interface (SIR) uses pulses of light to transmit data at a rate of 115.2 kilobits per second over distances of 1 to 3 meters. Magic Beam, which modulates a beam of light, uses less power to transmit less data over a longer distance 38.4 Kbps and 4 meters, respectively. Magic Beam is more like an FM receiver. Magic Beam is modulatable for different channels for having multiple communications going on in one area. Dual mode hardware supports both standards, similar to having AM and FM on one radio.

To improve location determination when indoors, a satellite GPS receiver 614 is embedded in the cell phone or cell phone charger and activated when connected to a power outlet.

Wrist Watch Module

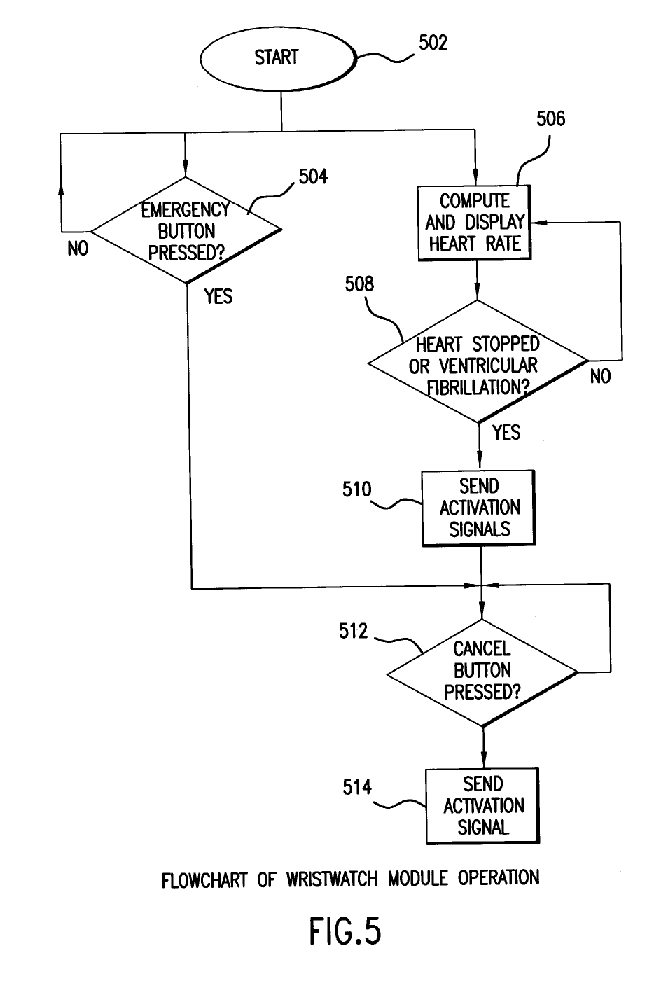

Referring now to FIG. 4, a pulse sensing circuit 402 generates a signal that correlates to the beating heart. Two common ways of automatic detection of heartbeats are through electrocardiography (ECU) or blood pressure sensing. Heart monitoring through ECU is accomplished by measuring electrical potentials generated by the heart on the surface of the body using two or more electrodes typically placed on the chest. The ECU produces distinguishable features corresponding to the contraction and filling phases of the chambers of the heart thus allowing heart rate to be computed. Heart rate, expressed usually in beats per minute, is computed by taking the inverse of average beat-to-beat interval over several beats. ECG-computed heart rate can be measured with a wrist worn device although with less accuracy because of a weaker signal. However, a wrist worn device would be the preferred implementation for the greater convenience during daily use. Watches providing the heart rate information are commercially available from manufacturers (such as “POLAR”) today.

Blood pressure also produces a distinct waveform when monitored continuously. A continuous blood pressure monitoring can be performed using tonometric sensors held with constant pressure onto the radial artery. The tonometric sensor measures the outward force of the pulse on the artery, thereby allowing heart rate measurement. In this method, a watch is worn on the palm side of the wrist, or the sensor is placed in the watch band.

Heart Rate Monitoring

The heart rate monitoring is controlled by the microprocessor 410 of the wristwatch module 400. The raw ECG or blood pressure signal from the pulse sensor 402 is first sent to a peak detection circuit 404. The peak detection circuit 404 is implemented using a comparator circuit (on the amplified signal), which in turn sends send a signal to the microprocessor 410 if a peak is detected.

Another method that is used is sampling the signal with an analog-to-digital converter and performing peak detection using the microprocessor 410. The microprocessor 410 computes heart rate from the detected beats and displays the result on an LCD display 414. Heart rate is computed by first averaging the time intervals of N number of last consecutive peaks. Taking the reciprocal of the average provides the heart rate over N beats. Heart rate is computed only if the sensor 402 is worn properly; otherwise, false alarms might be triggered. A monitor using ECG can measure impedance to ensure that the sensor 402 is worn correctly before generating the signal. The pulse sensor 402 with a tonometric blood pressure sensor does not produce a signal if a minimum force is not detected.

The heart monitor initiates an emergency call with one or more of the following situations:

1) Heart stoppage (defined as >3 seconds without a pulse)

2) Ventricular fibrillation (unsynchronized electrical activity detectable pulse for >3 seconds)

3) Severe bradycardia (low heart rate<20 bpm)

4) Ventricular flutter (hr>240 bpm)

5) Emergency button pressed for more than 3 seconds

The above parameters are examples only and can be modified accordingly.

Operation

Turning now generally to FIGS. 5-8, at step 702, an initiated emergency signal triggers a loud audible alarm (at step 706) on the wireless phone module 600. If a cancel button 408 is not pressed within a given time (as shown in step 708), the wireless phone module 600 initiates a 911 call (at step 714). If the cancel button 408 is pressed (at step 512) the alarm is disabled (at step 710), and the monitor shuts off allowing the wearer to correct the problem before it is turned on again. Pushing an emergency button 402 for four seconds manually places an emergency call.

FIG. 5 specifically shows a flow chart illustrating the operation of the wristwatch module. To save battery life, the wristwatch 400 transmits the radio signal only when an emergency is detected and/or when the cancel and/or emergency button is pressed. The watch transmits a signal strong enough to be detected by the wireless phone module 600 from a distance (e.g., 10 meters).

Wireless Phone Module/Operation

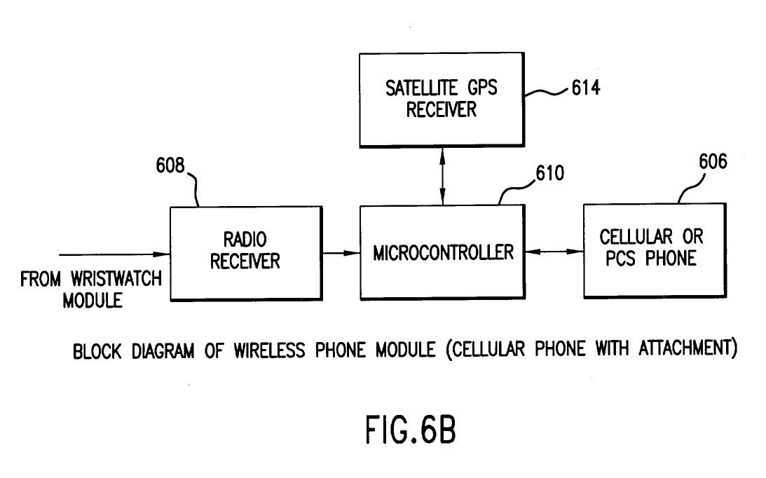

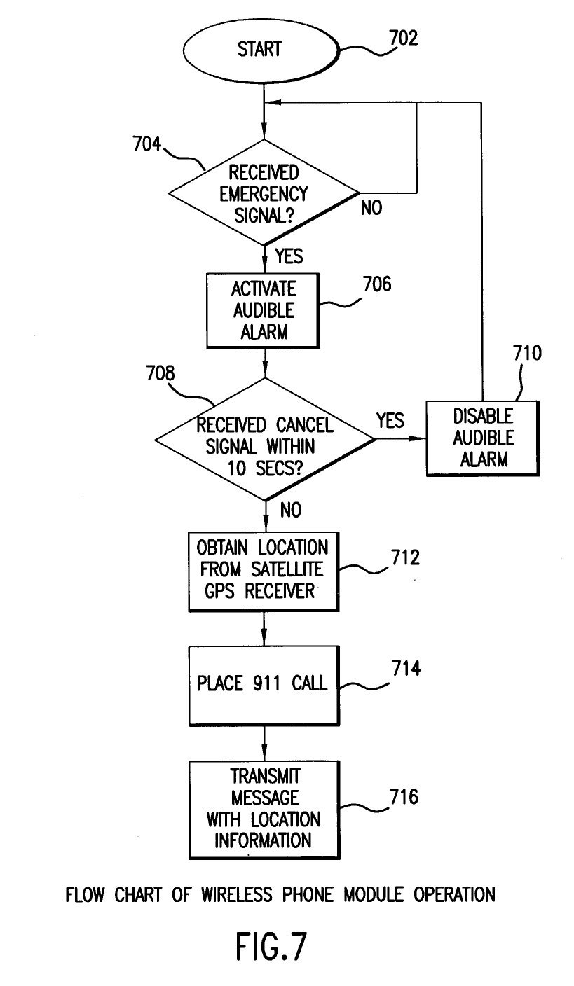

FIGS. 6a-6b specifically show the wireless phone module in detail. FIG. 7 specifically shows the operation of the wireless phone module. The wireless phone module 600 is responsible for placing the 911 call and computing a GPS location. The module 600 contains conventional cell phone components including a microcontroller 610, an LCD display 604, a DSP chip 620, a FLASH memory, a receiver/demodulator 602, a radio receiver 608, a GPS-enabled transmitter 606, a satellite GPS receiver 614, a keypad 612, a microphone 616, an A/D converter/bandpass filter/amplifier 618, a D/A converter/bandpass filter 622, and a speaker 624. The module 600 receives a radio signal from the wristwatch module 400 indicating that the heart monitor detected a heart problem or if the emergency button 406 is pressed. The module 600 generates a loud audible alarm for a given time (e.g., 10-30 seconds) allowing time for the wearer to cancel the emergency signal in case of a false alarm. If the cancel button 408 is not pressed, the emergency sequence is initiated. In another embodiment, the user defines the time period of the audible alarm.

In an indoor situation (with the phone plugged into the charger), the emergency sequence begins with the activation of the satellite GPS receiver 614 and computation of location coordinates. “911” is then automatically dialed (at step 714). Upon connection, at step 716, a digitally stored voice message is played stating that it is a medical emergency and that an ambulance is requested at the computed-location using the satellite GPS receiver 614. The message is repeated until the call is disconnected. The 911-operator receives location information both from the voice message and from the wireless service provider of the network on which the call was placed. During the emergency sequence, the loud audible alarm continues to sound. If the phone is not plugged in, the satellite GPS receiver 614 will not be activated and the voice message will not include location information. In another embodiment, the user customizes the outgoing message. For instance, the message is one or more of the following: “Please send an ambulance immediately to the location”; “Call my spouse at NPA-NXX-XXXX); “Please call my counsel”, etc.

When not in emergency mode, the wireless phone module 600 functions as a traditional cellular or PCS phone. In another embodiment, interfaces are available to which existing wireless phones are attached with a separate device to provide additional features. The interfaces include Bluetooth wireless interfaces, Serial Infrared communications interface (SIR), Magic Beam and other wired/wireless solutions.

In areas where no emergency services are available, the present invention is customizable through a non-illustrated setup screen so that only the loud signal is generated. Further, as better ways of locating an individual (indoors) become available, the present invention can be modified accordingly. Other methods (including optical detection of blood flow) have also been used to detect heartbeats advantageously with the present invention.

Non-Limiting Hardware Embodiments

The present invention can be realized in hardware, software, or a combination of hardware and software. A system according to a preferred embodiment of the present invention can be realized in a centralized fashion in one computer system, or in a distributed fashion where different elements are spread across several interconnected computer systems. Any kind of computer system (or other apparatus adapted for carrying out the methods described herein) is suitable. A typical combination of hardware and software could be a general purpose computer system with a computer program that, when being loaded and executed, controls the computer system such that it carries out the methods described herein.

The present invention is also embedded in a computer program product (for controlling the microprocessor 410 and microcontroller 610), which comprises all the features enabling the implementation of the methods described herein, and which—when loaded in a computer system—is able to carry out these methods. Computer program means or computer program in the present context mean any expression, in any language, code or notation, of a set of instructions intended to cause a system having an information processing capability to perform a particular function either directly or after either or both of the following a) conversion to another language, code or, notation; and b) reproduction in a different material form.

Each computer system may include, inter alia, one or more computers and at least a computer readable medium allowing a computer to read data, instructions, messages or message packets, and other computer readable information from the computer readable medium. The computer readable medium may include non-volatile memory, such as ROM, Flash memory, Disk drive memory, CD-ROM, and other permanent storage. Additionally, a computer medium may include, for example, volatile storage such as RAM, buffers, cache memory, and network circuits. Furthermore, the computer readable medium may comprise computer readable information in a transitory state medium such as a network link and/or a network interface, including a wired network or a wireless network, that allow a computer to read such computer readable information.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the appended claims cover any and all such applications, modifications, and embodiments within the scope of the present invention.

What is claimed is:

1. A method on a user unit of detecting cardiac arrest and automatically alerting emergency personnel of a wearer's location, the method comprising:
detecting at least one dangerous heart abnormality based on measuring a user's current pulse; and
generating an emergency signal to trigger a wireless module, wherein the wireless module emits an audible alarm for a given time, and if the alarm is not disabled within the given time, the wireless module initiates an emergency call for determining the geographic coordinates of the wireless module.

2. The method of claim 1, further comprising:
transmitting a predefined message after initiating the emergency call.

3. The method of claim 2, further comprising:
repeating the predefined message until the emergency call is disconnected.

4. The method of claim 1, wherein the emergency signal is a wireless signal selected from a group of wireless signals consisting of Bluetooth, Infra Red and 900 MHZ.

5. The method of claim 2, wherein the geographic coordinates are computed by a wireless network provider.

6. The method of claim 2, wherein the geographic coordinates are computed by activating a satellite global positioning system (GPS) receiver coupled to the wireless module.

7. The method of claim 6, wherein the wireless module receives the geographic coordinates from the GPS receiver.

8. The method of claim 6, further comprising:
requesting an emergency vehicle to the geographic coordinates.

9. The method of claim 1, wherein the dangerous heart abnormality is one of a heart stoppage, a ventricular fibrillation, bradycardia with a heart rate of less than 20 bpm, and a ventricular flutter.

10. The method of claim 1, wherein the user unit is a wrist watch.

11. A system for detecting cardiac arrest and automatically alerting emergency personnel of a wearer's location, the system comprising:
a first module for detecting at least one dangerous heart abnormality and generating an emergency signal; and
a second wireless module in communication with the first module for receiving the emergency signal therefrom, wherein:
the wireless module emits an audible alarm for a given time, and if the alarm is not disabled within the given time, the wireless module initiates an emergency call for determining the geographic coordinates of the wireless module.

12. The system of claim 11, wherein:
the first module contains a transmitter for communicating with the second module; and
the second module is a cell phone containing a receiver for communicating with the first module.

13. The system of claim 11, wherein:
the first module includes a cancel button for disabling the alarm; and
the second wireless module includes a satellite GPS receiver for computing the location's coordinates if the cancel button is not pressed within the given time.

14. The system of claim 11, wherein the second wireless module includes a battery charger integrated with the satellite GPS receiver.

15. The system of claim 11, further comprising:
a charger having a receiver coupled to the second wireless module.

16. The system of claim 13, further comprising:
means for transmitting a message with the computed coordinates.

17. A computer readable medium comprising computer instructions for performing a method on a user unit of detecting cardiac arrest and automatically alerting emergency personnel of a wearer's location, comprising:
detecting at least one dangerous heart abnormality based on measuring a user's current pulse; and
generating an emergency signal to trigger a wireless module, wherein the wireless module emits an audible alarm for a given time, and if the alarm is not disabled within the given time, the wireless module initiates an emergency call for determining the geographic coordinates of the wireless module.

18. The computer readable medium of claim 17, further comprising instructions for:
transmitting a predefined message after initiating the emergency call.

19. The computer readable medium of claim 18, further comprising instructions for:
repeating the predefined message until the emergency call is disconnected.

20. The computer readable medium of claim 17, wherein the emergency signal is a wireless signal selected from a group of wireless signals consisting of Bluetooth, Infra Red and 900 MHZ.

21. The computer readable medium of claim 18, wherein the geographic coordinates are computed by a wireless network provider.

22. The computer readable medium of claim 18, wherein the geographic coordinates are computed by activating a satellite global positioning system (GPS) receiver coupled to the wireless module.

23. The computer readable medium of claim 22, wherein the wireless module receives the geographic coordinates from the GPS receiver.

24. The computer readable medium of claim 17, wherein the dangerous heart abnormality is one of a heart stoppage, a ventricular fibrillation, bradycardia with a heart rate of less than 20 bpm, and a ventricular flutter.
